(19) United States
(12) Patent Application Publication
(10) Pub. No.: US 2003/0077667 A1
(43) Pub. Date: Apr. 24, 2003

(54) Methods and compounds for disruption of CD40R/CD40L signaling in the treatment of alzheimer's disease

(76) Inventors: Jun Tan, Tampa, FL (US); Terrence C. Town, Tampa, FL (US); Michael Mullan, Tampa, FL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/218,004

(22) Filed: Aug. 12, 2002

Related U.S. Application Data
(63) Continuation-in-part of application No. 09/585,058, filed on Jun. 1, 2000.
(60) Provisional application No. 60/137,016, filed on Jun. 1, 1999.
(60) Provisional application No. 60/311,115, filed on Aug. 10, 2001.

(51) Int. Cl. G01N 33/53; G01N 33/567; A61K 39/395

(52) U.S. Cl. 435/7.2; 424/144.1

(57) ABSTRACT

The subject invention provides methods of treating amyloidogenic diseases, comprising the administration of therapeutically effective amounts of a composition comprising a carrier and an agent that interferes with the interaction of CD40L and CD40R to an individual afflicted with an amyloidogenic disease. Also provided are methods and/or assay systems for the identification of compounds or other small molecules capable of disrupting the CD40R/CD40L signaling pathway.

### CROSS-REFERENCE TO RELATED APPLICATION(S)

The subject application is also a continuation-in-part of U.S. patent application Ser. No. 09/585,058, filed Jun. 1, 2001, pending, which claims priority to U.S. Provisional Application Serial No. 60/137,016, filed Jun. 1, 1999. The present application also claims priority to U.S. Provisional Application 60/311,115, filed Aug. 10, 2001, which is hereby incorporated by reference herein in its entirety, including any figures, tables, nucleic acid sequences, amino acid sequences, or drawings.

### BACKGROUND OF THE INVENTION

Deposition of β-amyloid (Aβ) in brain is a defining feature of Alzheimer's disease (AD), and there is evidence that activation of inflammatory pathways is important in the pathogenesis of the disease. With age, transgenic mice that overexpress the “Swedish” mutant amyloid precursor protein (Tg APP_{sw}, line 2576) show markedly elevated levels of cortical deposited Aβand gliosis. CD40 (CD40R) is a key immunoregulatory molecule, and we have previously shown that ligation of CD40R with its cognate ligand, CD40L, is required for triggering pro-inflammatory microglial activation induced by Aβ peptides.

Alzheimer's disease (AD) is the most common progressive dementing illness, and is neuropathologically characterized by deposition of the 40 to 42 amino acid β-amyloid peptide (Aβ) (proteolytically derived from the amyloid precursor protein, APP) as senile plaques. Concomitant with Aβ deposition there exists robust activation of inflammatory pathways in AD brain, including production of pro-inflammatory cytokines and acute-phase reactants in and around Aβ deposits (McGeer et al., “Inflammation in the brain in Alzheimer's disease: Implications for therapy,” J. Leukocyte Biol. (1999) 65:409-15; McGeer et al., “The importance of inflammatory mechanisms in Alzheimer's disease,” Exp. Gerontol. (1998) 33:371-8; Rogers et al., “Inflammation and Alzheimer's disease pathogenesis,” Neurobiol. Aging (1996) 17:681-6). Activation of the brain's resident innate immune cells, the microglia, is thought to be intimately involved in this inflammatory cascade, as reactive microglia produce proin-flammatory cytokines such as tumor necrosis factor alpha (TNF-α) and interleukin-1β, which (at high levels) promote neurodegeneration (Rogers et al., “Inflammation and Alzheimer's disease pathogenesis,” Neurobiol. Aging (1996) 17:681-6; Meda et al., “Activation of microglial cells by beta-amyloid and interferon-gamma,” Nature (1995) 374:647-50; Barger et al., “Microglial activation by Alzheimer amyloid precursor protein and modulation by apolipoprotein E,” Nature (1997) 388:878-81). Epidemiological studies have shown that patients using non-steroidal anti-inflammatory drugs (NSAIDs) have as much as 50% reduced risk for AD (Rogers et al., “Inflammation and Alzheimer's disease pathogenesis,” Neurobiol. Aging (1996) 17:681-6; Stewart et al., “Risk of Alzheimer's Disease and Duration of NSAID Use,” Neurology (1997) 48:626-32), and post-mortem evaluation of AD patients who underwent NSAID treatment has demonstrated that risk reduction is associated with diminished numbers of activated microglia (Mackenzie et al., “Nonsteroidal anti-inflammatory drug use and Alzheimer-type pathology in aging,” Neurology (1998) 50:986-90). Further, when transgenic mice that overexpress the “Swedish” APP mutation (Tg APP_{sw}) are given an NSAID (ibuprofen), these animals show reduction in Aβ deposits, astrocytosis, and dystrophic neurites correlating with decreased microglial activation (Lim et al., “Ibuprofen suppresses plaque pathology and inflammation in a transgenic mouse model for Alzheimer's disease,” J. Neurosci. (2000) 20:5709-14).

However, recent studies have indicated that the relationship between microglial activation and promotion of AD-like pathology is not straightforward, as some forms of microglial activation appear to mitigate this pathology. Schenk et al. have shown that immunization of the PDAPP mouse model of AD with Aβ₁₋₄₂ results in marked reduction of Aβ deposits, and atypical punctate structures containing Aβ that resembled activated microglia were found in brains of these mice, suggesting that immunization activates microglia to phagocytose Aβ (Schenk et al., “Immunization with beta-amyloid attenuates Alzheimer-disease-like pathology in the PDAPP mouse,” Nature (1999) 400:173-7). This hypothesis was further supported ex vivo, where microglia were shown to clear deposited Aβ that was opsonized by anti-Aβ antibodies (Bard et al, “Peripherally administered antibodies against amyloid betapeptide enter the central nervous system and reduce pathology in a mouse model of Alzheimer disease,” Nat. Med. (2000) 6:916-19). Similar prophylactic effects of Aβ₁₋₄₂ immunization have now been independently observed in other transgenic mouse models of AD (Morgan et al., “A beta peptide vaccination prevents memory loss in an animal model of Alzheimer's disease,” Nature (2000) 408:982-5; Janus et al., “A beta peptide immunization reduces behavioural impairment and plaques in a model of Alzheimer's disease,” Nature (2000) 408:979-82), and in vivo visualization has shown that application of anti-Aβ antibody to PDAPP mouse brain results in rapid Aβ plaque clearance associated with marked local microglial activation (as measured by lectin immunoreactivity) (Bacskai et al., “Imaging of amyloid-beta deposits in brains of living mice permits direct observation of clearance of plaques with immunotherapy,” Nat. Med. (2001) 7:369-72). Finally, bigenic mice that overexpress human APP and transforming growth factor β1 also demonstrate reduced parenchymal Aβ deposition associated with an increase in microglia positive for the F4/80 antigen (Wyss-Coray et al., “TGF-betal promotes microglial amyloid-beta clearance and reduces plaque burden in transgenic mice,” Nat. Med. (2001) 7:612-18).

CD40 is a ˜45 kDa key immunoregulatory molecule, which plays a critical role in immune cell activation. In the periphery, ligation of B cell CD40R promotes B cell proliferation after antigenic challenge, resulting in differentiation into antibody-secreting plasma cells. Blockade of the CD40R-CD40 ligand (CD40L) interaction in vivo inhibits activated T cell-dependent interleukin-12 secretion by antigen presenting cells (Grewal et al., “Requirement for CD40 ligand in costimulation induction, T cell activation, and experimental allergic encephalomyelitis,” Science (1996) 273:1864-7; Stuber et al., “Blocking the CD40L-CD40interaction in vivo specifically prevents the priming of T helper 1 cells through the inhibition of interleukin 12 secretion,” J. Exp. Med. (1996) 183:693-8).

We and others have shown that CD40is expressed on cultured microglia at low levels, and CD40R expression is markedly enhanced on these cells by the pro-inflammatory cytokine interferon-γ as well as Aβ (Carson et al., “Mature microglia resemble immature antigen-presenting cells,” Glia (1998) 22:72-85; Tan et al., “Activation of microglial cells by the CD40pathway: relevance to multiple sclerosis,” J. Neuroimmunol. (1999) 97:77-85; Tan et al., “Microglial activation resulting from CD40-CD40L interaction after beta-amyloid stimulation,” Science (1999) 286:2352-55). Aβ and CD40L synergistically stimulate microglia to secrete TNF-α, resulting in induction of neuronal injury in vitro, effects that are not observed in the presence of low levels of Aβ alone (Tan et al., “Microglial activation resulting from CD40R-CD40L interaction after beta-amyloid stimulation,” Science (1999) 286:2352-55). Further, interruption of CD40R-CD40L signaling in Tg APP_{sw} mice mitigates hyper-phosphorylation of the microtubule-associated protein tau (Tan et al., “Microglial activation resulting from CD40R-CD40L interaction after beta-amyloid stimulation,” Science (1999) 286:2352-55), a known marker of the pathogenic neuronal pre-tangle stage in AD brain. Additionally, in AD brain, CD40R expression is markedly increased on activated microglia and in senile plaques (Togo et al., “Expression of CD40in the brain of Alzheimer's disease and other neurological diseases,” Brain Res. (2000) 885:117-21). Recently, expression of CD40L and its receptor, CD40R, has been found in and around β-amyloid plaques in AD brain (Calingasan et al., “Identification of CD40ligand in Alzheimer's disease and in animal models of Alzheimer's disease and brain injury,” Neurobiol. Aging (2002) 23:31-9; Togo et al., “Expression of CD40 in the brain of Alzheimer's disease and other neurological diseases,” Brain Res. (2000) 885:117-21).

There is mounting evidence that products of the inflammatory process in AD brain exacerbate AD pathology. Many of these inflammatory proteins and acute phase reactants such as alpha-1-antichymotrypsin, transforming growth factor β, apolipoprotein E and complement factors are produced by activated glia, are localized to Aβ plaques, and have been shown to promote Aβ plaque “condensation” or maturation (Nilsson et al., “Alpha-1-antichymotrypsin promotes beta-sheet amyloid plaque deposition in a transgenic mouse model of Alzheimer's disease,” J. Neurosci. (2001) 21:1444-51; Harris-White et al., “Effects of transforming growth factor-beta (isoforms 1-3) on amyloid-beta deposition, inflammation, and cell targeting in organotypic hippocampal slice cultures,” J. Neurosci. (1998) 18:10366-74; Styren et al., “Expression of differential immune factors in temporal cortex and cerebellum: the role of alpha-1-antichymotrypsin, apolipoprotein E, and reactive glia in the progression of Alzheimer's disease,” J. Comp. Neurol. (1998) 396:511-20; Rozemuller et al., “A4 protein in Alzheimer's disease: primary and secondary cellular events in extracellular amyloid deposition,” J. Neuropathol. Exp. Neurol. (1989) 48:674-91). Further, there is evidence that activated microglia in AD brain, instead of clearing Aβ, are pathogenic by promoting Aβ fibrillogenesis and consequent deposition as senile plaque (Frackowiak et al., “Ultrastructure of the microglia that phagocytose amyloid and the microglia that produce beta-amyloid fibrils,” Acta Neuropathol. (Berl.) (1992) 84:225-33; Wegiel et al., “Microglia cells are the driving force in fibrillar plaque formation, whereas astrocytes are a leading factor in plague degradation,” Acta Neuropathol. (Berl.) (2000) 100:356-64).

### BRIEF SUMMARY OF THE INVENTION

The subject invention provides methods of treating neuronal inflammation, brain injury, tauopathies, or an amyloidogenic diseases, comprising the administration of therapeutically effective amounts of a composition comprising a carrier and an agent that interferes with the interaction of CD40L and CD40R to an individual afflicted with an amyloidogenic disease. Also provided are methods and/or assay systems for the identification of compounds or other small molecules capable of disrupting the CD40R/CD40L signaling pathway.

The subject invention provides a method of testing a compound suspected of modulating the CD40L/CD40R signaling pathway by interfering with CD40L/CD40R signaling pathway comprising: contacting a first sample of cells with CD40 ligand and measuring an inflammatory response; contacting a second sample of cells with a compound and CD40 ligand, and measuring an inflammatory response; comparing said inflammatory response of said first sample of cells with said inflammatory response of said second sample of cells. In this aspect of the invention, compounds modulate the CD40L/CD40R signaling pathway by interfering with the association of CD40L and CD40R, by interfering with components of the signaling pathway upstream or downstream of the CD40L/CD40R interaction, or by interfering with the trimerization of CD40R. In some aspect of the invention, compounds or small molecules that interfere with TNF receptor-associated factors (TRAFs) are contemplated.

In various embodiments, the cell samples are obtained from, or derived from, the central nervous system (CNS; e.g., biopsied materials obtained from humans), animal models, or peripheral sources. In some embodiments, the animal model cell samples comprise intact animals art recognized as models for Alzheimer's Disease or for the study of the CD40L/CD40R signaling pathway. The animal models may be transgenic or non-transgenic and non-limiting examples of these models include mice, worms, or flies; cells obtained from these animal models can be immortalized and cultured as cell lines. Cell samples can also include immortalized and non-immortalized cell lines derived from, for example, human, higher primate, primate, murine sources.

The subject invention also provides a method for testing a compound suspected of modulating the CD40L/CD40R signaling pathway by interfering with CD40L/CD40R signaling pathway comprising, said method comprising: a. contacting CNS cells with CD40 ligand and said compound and measuring an inflammatory response; b. contacting peripheral cells with CD40 ligand and said compound and measuring an inflammatory response; c. contacting CNS cells with a stimulator of the CD40 pathway and a compound and measuring an inflammatory response; d. contacting peripheral cells with a stimulator of the CD40 and said compound and measuring an inflammatory response; e. contacting CNS cells with an inhibitor of the CD40 pathway and said compound and measuring inflammatory response; f. contacting peripheral cells with an inhibitor of the CD40 pathway and said compound and measuring inflammatory response; and g. comparing said inflammatory responses, whereby the CD40 -modulating activity of said compound is tested.

In various embodiments, these methods measure the levels of various markers, or combinations of markers, associated with the inflammatory response by measuring the levels of one or more markers. Cytokine markers can be selected from the group consisting of tumor necrosis factor, interleukin 1, interleukin 6, interleukin 12, interleukin 18, macrophage inflammatory protein, macrophage chemoattractant protein, granulocyte-macrophage colony stimulating factor, macrophage colony stimulating factor and various combinations of these cytokines. Alternatively, the methods measure levels or amounts of one or more markers selected from the group consisting of glutamate release, nitric oxide production, nitric oxide synthase, superoxide, superoxide dismutase and various combinations of these markers. The methods set forth herein can also measure a major histocompatibility complex molecule, CD45, CD11b, integrins, or a cell surface molecule as a marker of the inflammatory response. Yet other embodiments measure levels, amounts, or deposition of proteins on cells wherein said proteins are selected from the group consisting of Aβ, β-amyloid precursor protein, a fragment of a β-amyloid precursor protein, and combinations of these proteins. Stimulators and inhibitors according to the subject invention can be agonistic or antagonistic antibodies.

The subject invention also provides a method for testing a compound for its ability to modulate CD40L/CD40R interactions comprising contacting a CD40 receptor and a CD40 ligand with said compound and measuring the binding of said CD40 receptor with said CD40 ligand. In these types of assays, compounds can bind to CD40L or CD40R. The compounds can be small molecules or antibodies specific for CD40L or CD40R.

The subject invention also provides methods of conducting in vivo assays for compounds that are capable of modulating the CD40/CD40R signaling pathway comprising administering to an animal model, or a human, an agent or compound that modulates the signaling pathway, and measuring an the animal's responsiveness to the compound. In various embodiments, the method can be practiced with agents as described supra or soluble CD40L, an antibody against CD40 that inhibits the CD40 pathway, an antibody against CD40 ligand that inhibits the CD40 pathway, an antibody against CD40 that stimulates the CD40 pathway, an antibody against CD40 ligand that stimulates the CD40 interaction with CD40 ligand, a compound that blocks the CD40 pathway, a compound that interrupts the CD40 interaction with CD40 ligand, a compound that stimulates the CD40 pathway, or a compound that stimulates the CD40 interaction with CD40 ligand. Animals can be examined for improvements in conditions described supra or for improvements in β-amyloid deposition, soluble β-amyloid, inflammatory markers, microglial activation, astrocytic activation, neuronal apoptosis, neuronal necrosis, brain injury, tau phosphorylation, or tau paired helical filaments.

Also provided is a non-human transgenic animal model comprising one or more of the following: transgenic amyloid-precursor protein, overexpressed transgenic presenilin protein, overexpressed transgenic CD40 receptor, overexpressed transgenic CD40 ligand, and/or tau protein or mutants of the tau protein.

### DESCRIPTION OF THE FIGSURES

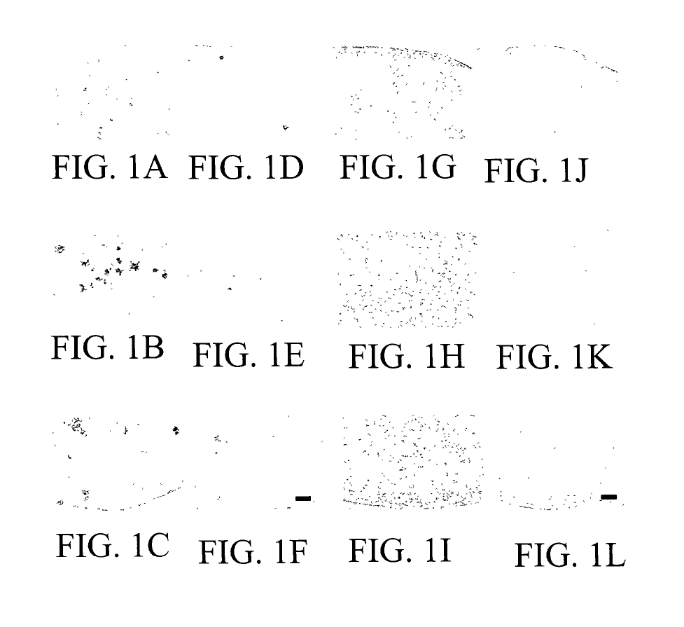

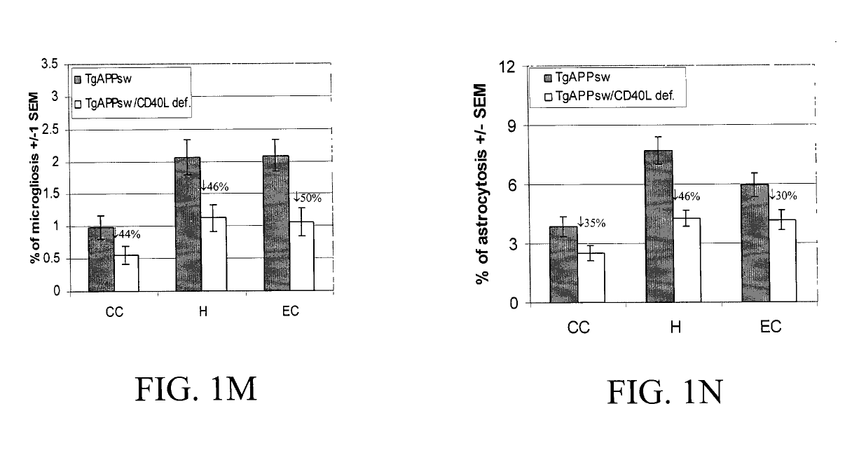

FIGS. 1a-1n: Microgliosis and astrocytosis are reduced in TgAPP/CD40L def. mice by 16 months of age. Panels are representative 10x bright-field photomicrographs. a-f, mouse brain sections stained with anti-CD11b antibody; left column represents sections from TgAPP_{sw} mice, and sections shown on the right were taken from TgAPP_{sw}/CD40L def. mice. Panels a and d represent cingulate cortices (CC); b and e, hippocampi (H); and c and If enthorinal cortices (EC). g-l, mouse brain sections stained with anti-GFAP antibody; left column represents sections from TgAPP_{sw} mice, and sections shown on the right were taken from Tg APP_{sw}/CD40L def. mice. Panels g and j represent CC; h and k, H; and i and l, EC. Scale bar denotes 100 μm (calculated for each panel). m, percentage of microgliosis and n, astrocytosis (mean±1 SEM) were calculated by quantitative image analysis, and percentage reduction for each brain region is indicated. The t-Test for independent samples revealed significant between-groups differences for each brain region examined in m and n (p<0.001 for each comparison).

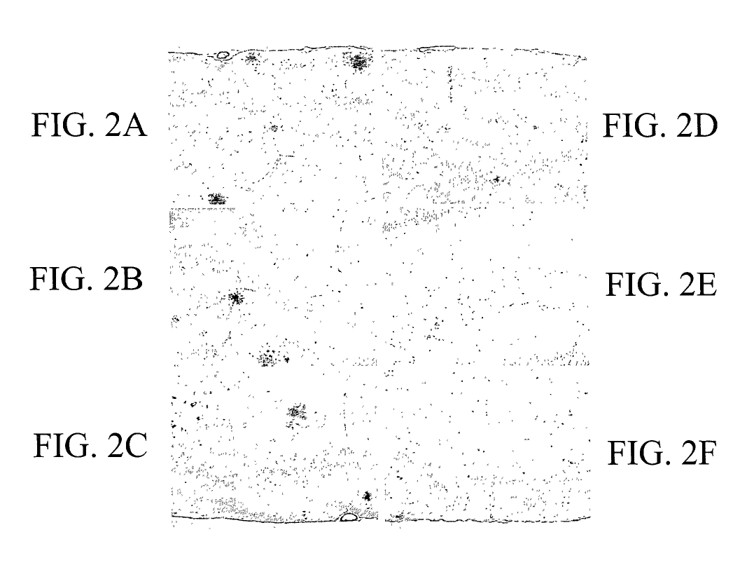

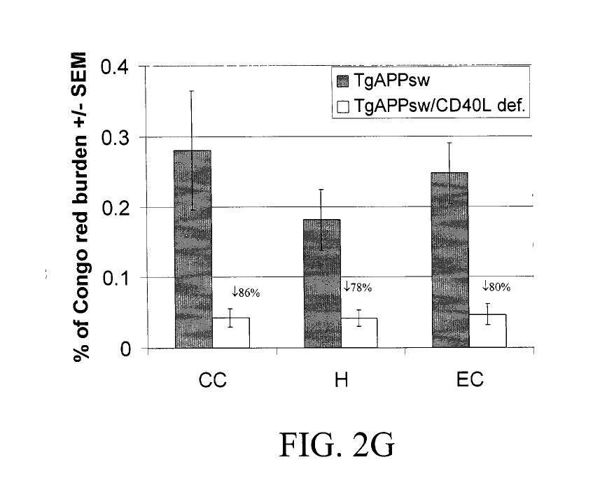

FIGS. 2a-2g: Congophilic amyloid deposits are markedly reduced in TgAPP_{sw}/CD40L def. mice by 16 months of age. Panels a-f are representative 10× bright-field photomicrographs of mouse brain sections stained with congo red. The left column represents sections from TgAPP_{sw} mice, and sections shown on the right were taken from TgAPP_{sw}/CD40L def. mice. Panels a and d represent cingulate cortices (CC); b and e, hippocampi; and c and f enthorinal cortices (EC). Scale bar denotes 100 μm (calculated for each panel). Each of the left column panels show abundant congo red-positive amyloid deposits compared to the corresponding right panels. g, Congo red burden was calculated by quantitative image analysis (mean±1 SEM), and percentage reduction for each brain region is indicated. The t-Test for independent samples revealed significant between-groups differences for each brain region examined (p<0.001 for each comparison).

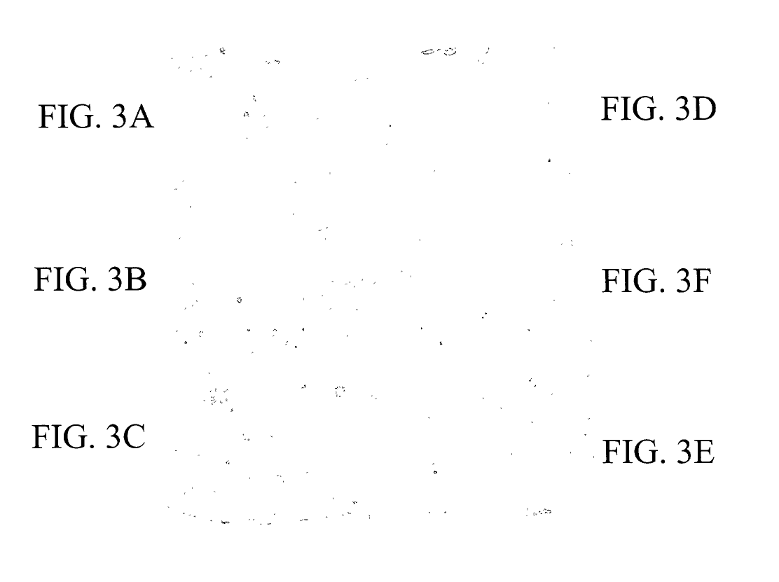

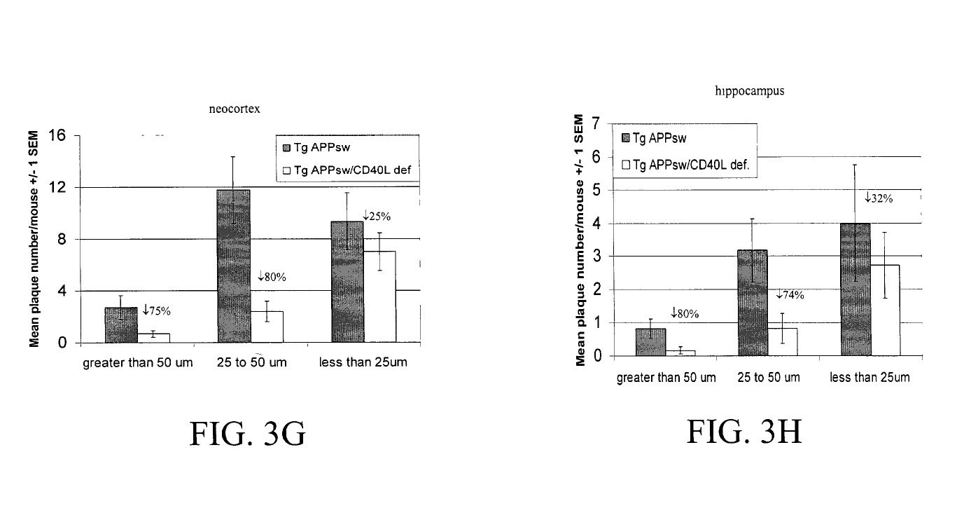

FIGS. 3a-3h: Morphometric analysis of Aβ plaques in TgAPP_{sw}/CD40L def. mice versus TgAPP_{sw} mice. Panels a-f are representative 10× bright-field photomicrographs of mouse brain sections (at 16 months of age) stained with anti-Aβ antibody. The left column represents sections from TgAPP_{sw} mice, and sections shown on the right were taken from TgAPP_{sw}/CD40L def. mice. Panels a and d represent cingulate cortices (CC); b and e, hippocampi (H); and c and f enthorinal cortices (EC). Scale bar denotes 100 μm (calculated for each panel). Note the increased number of large diameter Aβ plaques in each of the left columns compared to corresponding right columns. Quantitative morphometric analysis results (mean plaque subtype per mouse±1 SEM) are displayed for g, the neocortex and h, the hippocampus, and percentage reduction of plaques in TgAPP_{sw}/CD40L def. mice versus TgAPP_{sw} mice is indicated. For g and h, t-Test for independent samples revealed significantly fewer large (greater than 50 μm) and medium-sized (between 25 and 50 μm) Aβ plaques in TgAPP_{sw} /CD40L def. mice compared to TgAPP_{sw}mice (p<0.001 for each comparison).

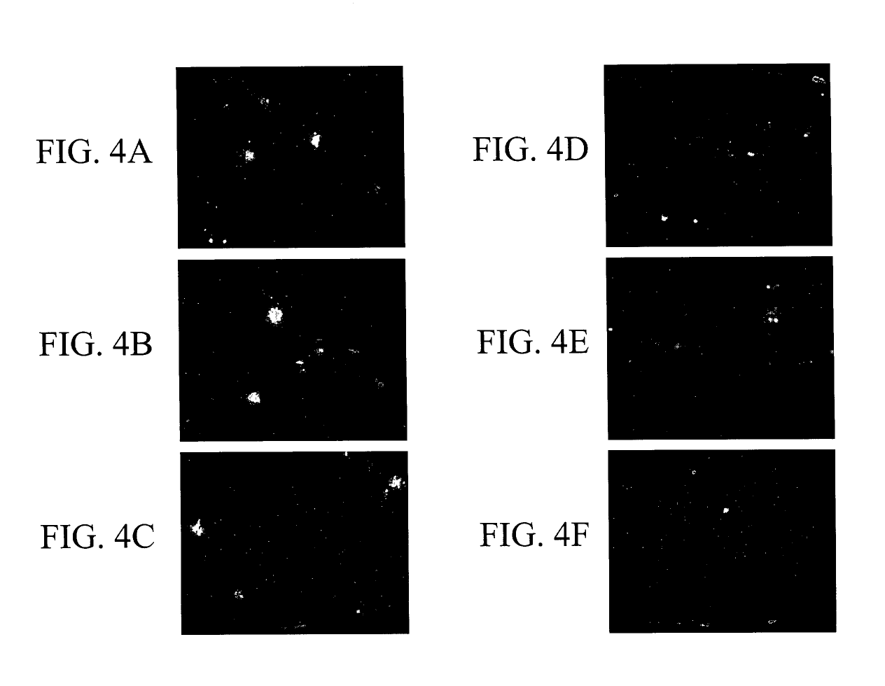

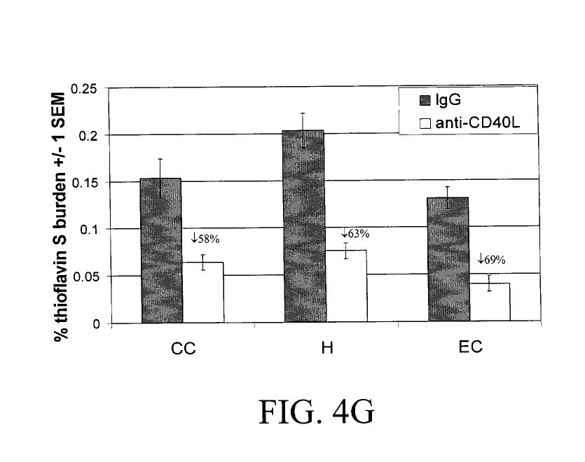

FIGS. 4a-4g: Reduced thioflavin S plaques in PSAPP mice treated with anti-CD40L antibody. Panels are 20× bright-field photomicrographs taken from 8-month-old PSAPP mice that received anti-CD40L antibody or isotype-matched control IgG antibody. a-f, mouse brain sections stained with thioflavin S; left column shows sections from isotype-matched IgG-treated mice, and sections shown in the right column were taken from anti-CD40L antibody-treated mice. Panels a and d were taken from cingulate cortices (CC); b and e, hippocampi (H); and c and f, entorhinal cortices (EC). g, percentages of thioflavin S-staining β-amyloid plaques (mean±1 SEM) were quantified by image analysis, and percentage reduction for each brain region is indicated. The t-Test for independent samples revealed significant between-groups differences for each brain region examined in g (p<0.001 for each comparison).

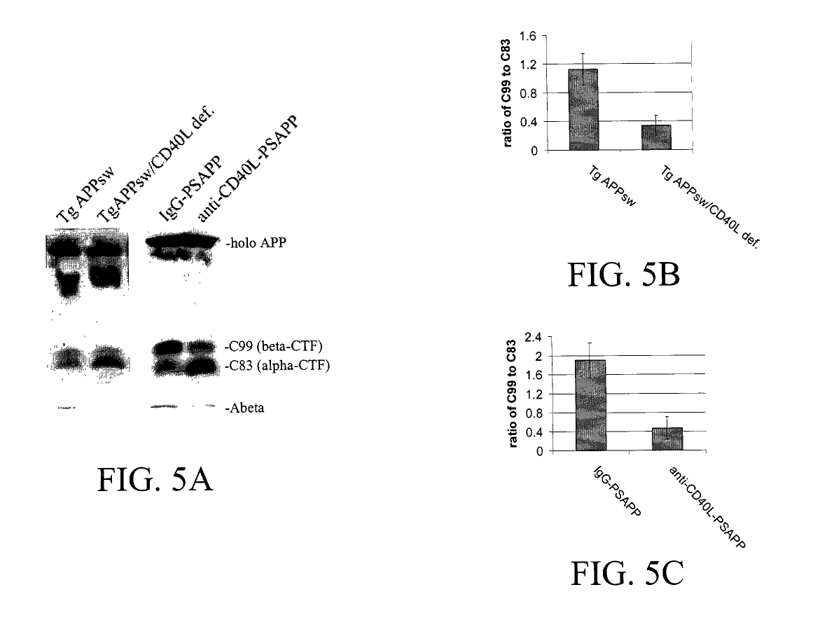

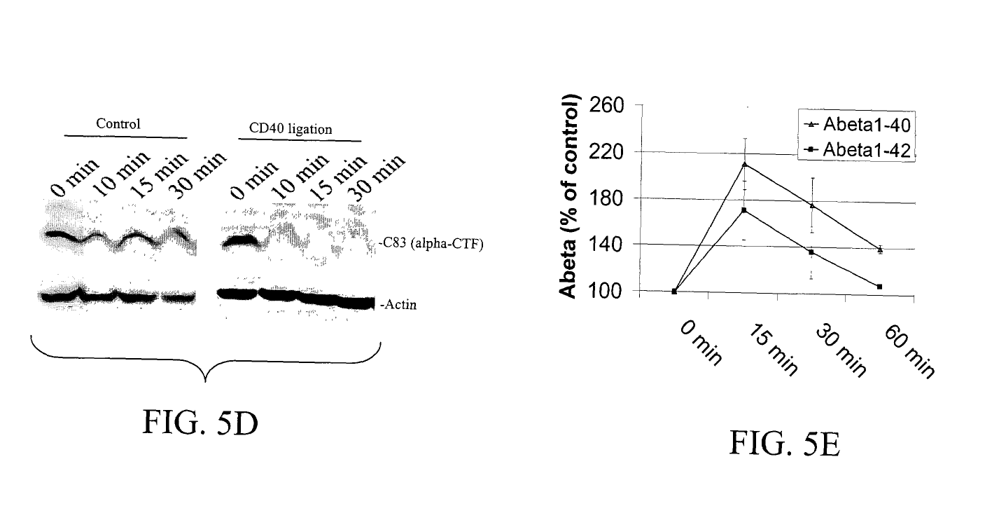

FIGS. 5a-5e: CD40L modulates APP processing in vivo and in vitro. Brain homogenates were prepared from 12-month-old Tg APP_{sw,} Tg APP_{sw}/CD40L deficient (def.), control IgG-treated PSAPP, and anti-CD40L antibody-treated PSAPP animals. Representative lanes are shown from each mouse group. a, Western immunoblot by antibody 369 against the cytoplasmic tail of APP reveals holo APP, and two bands corresponding to C99 (β-CTF) and C83 (α-CTF) as indicated (top panel). Antibody BAM-10 reveals Aβ species (lower panel). b and c, densitometry shows the ratio of C99 to C83, with n=5 for each mouse group. The t-Test for independent samples revealed significant differences for each comparison (p<0.001). Cell lysates and conditioned media were prepared from N2a cells over-expressing human APP and treated with 2 μg/mL of heat-inactivated CD40L (control) or CD40L protein (CD40 ligation) at the time points indicated. d, C-terminal fragments of APP were analyzed in cell lysates by Western immunoblot using antibody 369. Similar results were obtained with antibody 6687 or Chemicon polyclonal APP C-terminal antibody. e, Aβ₁₋₄₀ and Aβ₁₋₄₂ peptides were analyzed in human APP-overexpressing N2a cells by ELISA. Data are represented as percentage of Aβ peptide secreted after CD40 ligation relative to control protein treatment. ANOVA revealed a significant effect of incubation period on Aβ₁₋₄₀ and Aβ₁₋₄₂ (p<0.01) levels. Data shown are representative of three independent experiments.

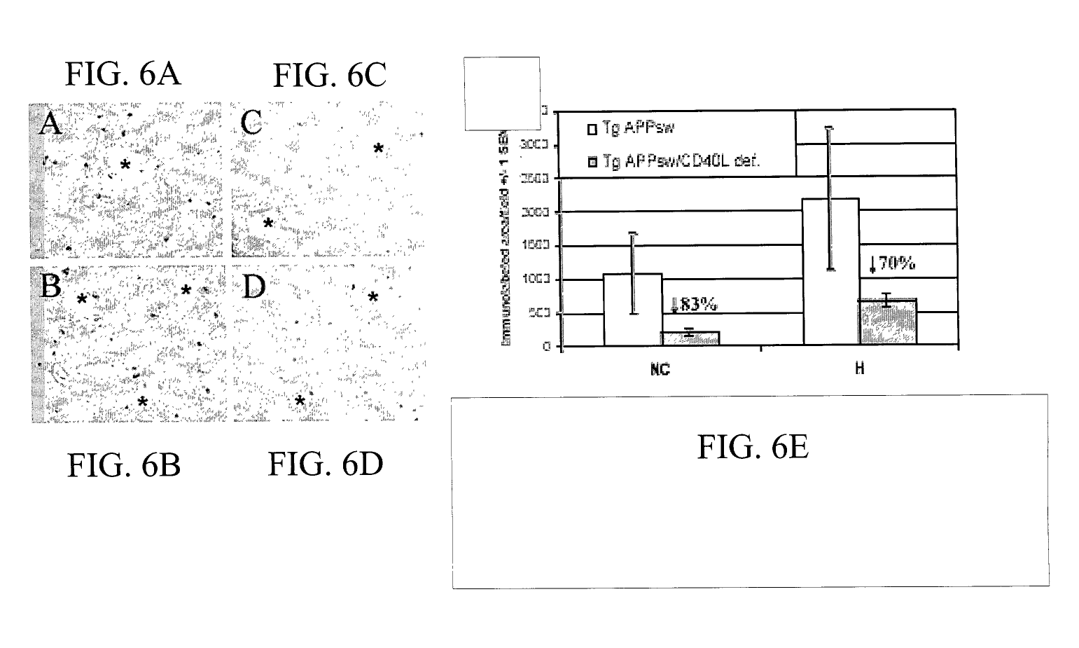

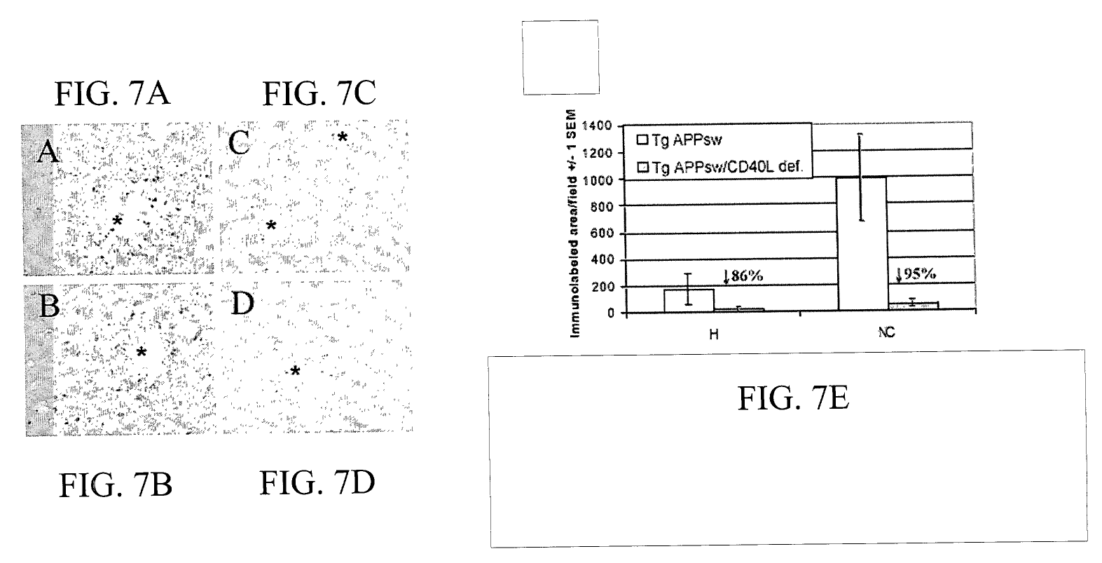

FIGS. 6A-6E. Phospho-tau in situ by antibody pS199. 40× photomicrographs (FIGS. 7A and 7B) were taken from 16-month-old Tg APP_{sw} mice (n=4) and FIGS. 7C and 7D are from age-matched Tg APP_{sw}/CD40L def. mice (n=5). FIGS. 7A and 7C are from the neocortex and FIGS. 7B and 7D are from the hippocampus. (*) indicates Aβ plaques. Quantitative analysis of pooled date is shown in FIG. 7E.

FIGS. 7A-7E. Phospho-tau in situ by antibody pS202. 40× photomicrographs (FIGS. 8A and 8B) were taken from 16-month-old Tg APP_{sw} mice (n=4) and FIGS. 8C and 8D are from age-matched Tg APP_{sw}/CD40L def. mice (n=5). FIGS. 8A and 8C are from the neocortex and FIGS. 8B and 8D are from the hippocampus. (*) indicates Aβ plaques. Quantitative analysis of pooled date is shown in FIG. 8E.

### DETAILED DESCRIPTION

The subject invention provides methods of treating neuronal inflammation, brain injury, tauopathies, or amyloidogenic diseases, comprising the administration of therapeutically effective amounts of a composition comprising a carrier and an agent that interferes with CD40L/CD40R signaling pathway to an individual afflicted with neuronal inflammation, brain injury, tauopathies, or an amyloidogenic disease. The phrase “interferes with CD40L/CD40R signaling pathway” can be construed as disrupting the binding or association of CD40L with its cognate receptor, e.g., CD40R or interfering with the trimerization of CD40R. Alternatively, the phrase can be construed as disrupting the signaling pathway upstream or downstream of CD40L/CD40R binding. Where tauopathies are to be treated, agents reduces the phosphorylation of the tau protein or mutants thereof.

CD40 ligand (CD40L) refers to native, recombinant or synthetic forms of the molecule. Native, recombinant, or synthetic forms of CD40L (termed CD40L variants [CD40LV]) can contain amino acid substitutions, additions, or deletions that do not affect the ability of the ligand to bind to the CD40receptor (CD40R); in certain embodiments CD40LV bind to CD40R, are unable to activate the CD40R, and block the binding of native CD40L (e.g., CD40L having the naturally occurring amino acid sequence and the ability to activate CD40R).

Nonlimiting examples of “tauopathies” include frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, disinhibition-dementia-parkinsonism-amytrophy complex, Pick's disease, or Pick's disease-like dementia.

“Amyloidogenic diseases” include, but not limited to, scrapie, transmissible spongioform encephalopathies (TSE's), hereditary cerebral hemorrhage with amyloidosis Icelandic-type (HCHWA-I), hereditary cerebral hemorrhage with amyloidosis Dutch-type (HCHWA-D), familial Mediterranean fever, familial amyloid nephropathy with urticaria and deafness (Muckle-Wells syndrome), myeloma or macroglobulinernia-associated idopathy associated with amyloid, familial amyloid polyneuropathy (Portuguese), familial amyloid cardiomyopathy (Danish), systemic senile amyloidosis, familial amyloid polyneuropathy (Iowa), familial amyloidosis (Finnish), Gerstmann-Staussler-Scheinker syndrome, medullary carcinoma of thyroid, isolated atrial amyloid, Islets of Langerhans, diabetes type II, and insulinoma. (Need exemplary tauopathies).

The phrase “therapeutically effective amounts” is to be construed as an amount of a composition that confers an improvement in the condition of an individual treated according to the methods taught herein. Non-limiting examples of such improvements for an individual include, improvements in quality of life and/or memory, reductions in the size and/or number of amyloid plaques, reduction in β-amyloid burden, reduction of congophilic β-amyloid deposits, reduction of reactive gliosis, microgliosis, and/or astrocytosis, an improvement in the symptoms with which an individual presented to a medical practitioner (e.g., reductions in the severity of symptoms with which the individual presented), or reduction of other β-amyloid associated pathologies.

An “agent that interferes with the interaction of CD40L and CD40R” includes, and is not limited to, soluble CD40R, antibodies that bind to CD40L and block its interaction with CD40R, antibodies that bind to CD40R and block ligand binding to the receptor, soluble CD40LV that bind to CD40R, but fail to activate the receptor, agents that reduce or inhibit the trimerization of CD40R, interfering RNA (dsRNA or RNAi) that suppresses or reduces the levels CD40R expression, antisense RNA to CD40R (in amounts sufficient to suppress or reduce the levels of CD40R expression), RNAi that reduces the levels or amounts of amyloid-β (Aβ) protein that is expressed and that block or suppresses/reduces the ability of Aβ to induce CD40R expression, antibodies that bind to Aβ and block or suppress/reduce its ability to induce CD40R expression. Antibodies that bind to CD40R can agonize or, preferably, antagonize the function of the receptor. In some embodiments, CD40L is rendered immunogenic according to methods known in the art and used to engender an immune response to native CD40L.

Methods of making soluble CD40L are known in the art (see for example U.S. Pat. No. 5,962,406 which is hereby incorporated by reference in its entirety) as are methods of interfering with CD40L/CD40R interactions (see U.S. Pat. No. 6,264,951, also hereby incorporated by reference in its entirety). Likewise, methods of mutagenizing receptor ligands and analyzing the effects of such mutagenesis on receptor ligand interaction is well known in the art and are described in the aforementioned U.S. patents.

Antisense technology can also be used to interfere with the CD40L/CD40R signaling pathway. For example, the transformation of a cell or organism with the reverse complement of a gene encoded by a polynucleotide exemplified herein can result in strand co-suppression and silencing or inhibition of a target gene, e.g., Aβ, CD40L, or CD40R. Therapeutic protocols and methods of practicing antisense therapies for the modulation of CD40R are well-known to the skilled artisan (see, for example, U.S. Pat. Nos. 6,197,584 and 6,194,150, each of which is hereby incorporated by reference in its entirety).

The ability to specifically inhibit gene function in a variety of organisms utilizing antisense RNA or dsRNA-mediated interference (RNAi or dsRNA) is well known in the fields of molecular biology (see for example C. P. Hunter, Current Biology [1999 ] 9:R440-442; Hamilton et al., [1999] Science, 286:950-952; and S. W. Ding, Current Opinions in Biotechnology [2000] 11:152-156, hereby incorporated by reference in their entireties). dsRNA (RNAi) typically comprises a polynucleotide sequence identical or homologous to a target gene (or fragment thereof) linked directly, or indirectly, to a polynucleotide sequence complementary to the sequence of the target gene (or fragment thereof). The dsRNA may comprise a polynucleotide linker sequence of sufficient length to allow for the two polynucleotide sequences to fold over and hybridize to each other; however, a linker sequence is not necessary. The linker sequence is designed to separate the antisense and sense strands of RNAi significantly enough to limit the effects of steric hindrances and allow for the formation of dsRNA molecules and should not hybridize with sequences within the hybridizing portions of the dsRNA molecule. The specificity of this gene silencing mechanism appears to be extremely high, blocking expression only of targeted genes, while leaving other genes unaffected. Accordingly, one method for treating amyloidogenic diseases according to the subject invention comprises the use of materials and methods utilizing double-stranded interfering RNA (dsRNAi), or RNA-mediated interference (RNAi) comprising polynucleotide sequences identical or homologous to CD40L and/or CD40R. The terms “dsRNAi”, “RNAi”, “iRNA”, and “siRNA” are used interchangeably herein unless otherwise noted.

RNA containing a nucleotide sequence identical to a fragment of the target gene is preferred for inhibition; however, RNA sequences with insertions, deletions, and point mutations relative to the target sequence can also be used for inhibition. Sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Alternatively, the duplex region of the RNA may be defined functionally as a nucleotide sequence that is capable of hybridizing with a fragment of the target gene transcript.

RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands); the promoters may be known inducible promoters such as baculovirus. Inhibition may be targeted by specific transcription in an organ, tissue, or cell type. The RNA strands may or may not be polyadenylated; the RNA strands may or may not be capable of being translated into a polypeptide by a cell's translational apparatus. RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (see, for example, WO 97/32016; U.S. Pat. Nos. 5,593,874; 5,698,425; 5,712,135; 5,789,214; and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no, or a minimum of, purification to avoid losses due to sample processing. The RNA may be dried for storage or dissolved in an aqueous solution. The solution may contain buffers or salts to promote annealing, and/or stabilization of the duplex strands.

Preferably and most conveniently, dsRNAi can be targeted to an entire polynucleotide sequence, such as the CD40R, CD40L, or Aβ. Preferred RNAi molecules of the instant invention are highly homologous or identical to the polynucleotides encoding CD40R, CD40L, or Aβ. The homology may be greater than 70%, preferably greater than 80%, more preferably greater than 90% and is most preferably greater than 95%.

Fragments of genes can also be utilized for targeted suppression of gene expression. These fragments are typically in the approximate size range of about 20 consecutive nucleotides of a target sequence. Thus, targeted fragments are preferably at least about 15 consecutive nucleotides. In certain embodiments, the gene fragment targeted by the RNAi molecule is about 20-25 consecutive nucleotides in length. In a more preferred embodiment, the gene fragments are at least about 25 consecutive nucleotides in length. In an even more preferred embodiment, the gene fragments are at least 50 consecutive nucleotides in length. Various embodiments also allow for the joining of one or more gene fragments of at least about 15 nucleotides via linkers. Thus, RNAi molecules useful in the practice of the instant invention can contain any number of gene fragments joined by linker sequences.

In yet other embodiments, the gene fragments can range from one nucleotide less than the full-length gene (X_{CD40L}=n−1; X_{CD40R}=n−1; or X_{Aβ}=n−1 wherein X is a given whole number fragment length and n is the number of nucleotides in the full length CD40L, CD40R, or Aβ sequence). Nucleotide sequences for CD40R, CD40L, and Aβ are known in the art and can be obtained from patent publications, public databases containing nucleic acid sequences, or commercial vendors. This paragraph is also to be construed as providing written support for any fragment length ranging from 15 consecutive polynucleotides to one nucleotide less than the full length polynucleotide sequence of CD40L, CD40R, or Aβ; thus, X_{CD40L}, X_{CD40R}, or X_{Aβ} can have a whole number value ranging from 15 consecutive nucleotides to one nucleotide less than the full length polynucleotide.

Accordingly, methods utilizing RNAi molecules in the practice of the subject invention are not limited to those that are targeted to the full-length polynucleotide or gene. Gene product can be inhibited with an RNAi molecule that is targeted to a portion or fragment of the exemplified polynucleotides; high homology (90-95%) or greater identity is also preferred, but not essential, for such applications.

In another aspect of the invention, the dsRNA molecules of the invention may be introduced into cells with single stranded (ss) RNA molecules which are sense or anti-sense RNA derived from the nucleotide sequences disclosed herein. Methods of introducing ssRNA and dsRNA molecules into cells are well-known to the skilled artisan and includes transcription of plasmids, vectors, or genetic constructs encoding the ssRNA or dsRNA molecules according to this aspect of the invention; electroporation, biolistics, or other well-known methods of introducing nucleic acids into cells may also be used to introduce the ssRNA and dsRNA molecules of this invention into cells.

In another embodiment of the invention, the subject invention provides methods for the treatment of internal organ diseases related to amyloid plaque formation, including plaques in the heart, liver, spleen, kidney, pancreas, brain, lungs and muscles comprising the administration of therapeutically effective amounts of a composition comprising a carrier and an agent that interferes with the CD40L/CD40R signaling pathway to an individual in need of such treatment.

In another embodiment, the present invention provides assays for identifying small molecules or other compounds capable of modulating CD40R/CD40L pathways. The assays can be performed in vitro using non-transformed cells, immortalized cell lines, recombinant cell lines, transgenic cells, transgenic cell lines, or transgenic animals and cells/cell lines derived therefrom. Transgenic animals suitable for use in the subject invention include transgenic worms, transgenic flies, transgenic mice. For in vitro assays, cells and cell lines can be of human or other animal origin. Specifically the assays can be used to examine the effects of small molecules or other compounds on with neuronal inflammation, brain injury, tauopathies, or an amyloidogenic disease. In such assays, the small molecules or other compounds are tested for the ability to elicit an improvement in the condition of an individual to be treated according to the methods taught herein. Thus, for example, cells are examined for decreased inflammation, other suitable changes in or markers that are followed by the skilled artisan. In another embodiment, the subject invention provides in vivo methods of identifying small molecules or other compounds capable of modulating CD40R/CD40L signaling pathways comprising the administration of such compounds to individuals (e.g., human volunteers or murine models (such as those taught herein)) and examining the individuals for an improvement in the condition of an individual treated according to the methods taught herein.

The subject invention also provides therapeutic compounds or small molecules and compositions comprising a carrier and said therapeutic compounds or small molecules. In certain embodiments, the carrier is a pharmaceutically acceptable carrier or diluent.

Compositions containing therapeutic compounds and/or small molecules can be administered to a patient in a variety of ways including, for example, parenterally, orally or intraperitoneally. Parenteral administration includes administration by the following routes: intravenous, intramuscular, interstitial, intra-arterial, subcutaneous, intraocular, intracranially, intraventricularly, intrasynovial, transepithelial, including transdermal, pulmonary via inhalation, opthalmic, sublingual and buccal, topical, including ophthalmic, dermal, ocular, rectal, and nasal inhalation via insufflation or nebulization.

Compounds or small molecules that are orally administered can be enclosed in hard or soft shell gelatin capsules, or compressed into tablets. Active compounds or small molecules can also be incorporated with an excipient and used in the form of ingestible tablets, buccal tablets, troches, capsules, sachets, lozenges, elixirs, suspensions, syrups, wafers, and the like. The pharmaceutical composition comprising the active compounds can be in the form of a powder or granule, a solution or suspension in an aqueous liquid or non-aqueous liquid, or in an oil-in-water or water-in-oil emulsion.

The tablets, troches, pills, capsules and the like can also contain, for example, a binder, such as gum tragacanth, acacia, corn starch or gelating, excipients, such as dicalcium phosphate, a disintegrating agent, such as corn starch, potato starch, alginic acid and the like, a lubricant, such as magnesium stearate, and a sweetening agent, such as sucrose, lactose or saccharin, or a flavoring agent. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens as preservatives, a dye and flavoring. Any material used in preparing any dosage unit form should be pharmaceutically pure and substantially non-toxic. In addition, the active compound can be incorporated into sustained-release preparations and formulations.

The active compounds can be administered to the CNS, parenterally or intraperitoneally. Solutions of the compound as a free base or a pharmaceutically acceptable salt can be prepared in water mixed with a suitable surfactant, such as hydroxypropylcellulose. Dispersions can also be prepared in glycerol, liquid polyethylene glycols, and mixtures thereof, and in oils. Under ordinary conditions of storage and use, these preparations can contain a preservative and/or antioxidants to prevent the growth of microorganisms or chemical degeneration.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It can be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size (in the case of a dispersion) and by the use of surfactants. The prevention of the action of microorganisms can be brought about by various antibacterial and anti-fungal agents, for example, parabens, chlorobutanol, phenol, sorbic acid, thimerosal, and the like. In many cases, it will be preferable to include isotonic agents, for example, sugars or sodium chloride.

Sterile injectable solutions are prepared by incorporating the active compound in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and any of the other ingredients from those enumerated above. In the case of sterile powders for the preparation of sterile injectable solutions, the preferred methods of preparation are vacuum drying and the freeze drying technique.

Pharmaceutical compositions which are suitable for administration to the nose or buccal cavity include powder, self-propelling and spray formulations, such as aerosols, atomizers and nebulizers.

The therapeutic compounds of this invention can be administered to a mammal alone or in combination with pharmaceutically acceptable carriers or as pharmaceutically acceptable salts, the proportion of which is determined by the solubility and chemical nature of the compound, chosen route of administration and standard pharmaceutical practice.

The compositions can also contain other therapeutically active compounds which are usually applied in the treatment of the diseases and disorders discussed herein. Treatments using the present compounds and other therapeutically active compounds can be simultaneous or in intervals.

### EXAMPLE 1

### Genetic Disruption of CD40R/CD40L in Mammals

Genetic disruption of CD40L in Tg APP_{sw} mice results in reduced activation of microglia and astrocytes. These changes are concomitant with reduced Aβ pathology, with the most notable diminution in mature congophillic β-amyloid plaques at 16 months of age by 77-85%. Correspondingly, large (greater than 50 μm) and medium-sized (between 25 and 50 μm) Aβ plaques are reduced by approximately the same amount in these animals. These data indicate that CD40R-CD40L signaling is important for the development of Aβ pathology.

Genetic disruption of CD40L in Tg APP_{sw} mice also results in reduced soluble and deposited Aβ levels, with up to 85% diminution, or more, of mature congophillic β-amyloid plaques. Correspondingly, large (greater than 50 μm) and medium-sized (between 25 and 50 μm) β-amyloid plaques are diminished by a comparable magnitude in these animals. These changes are concomitant with reduced brain inflammation as measured by reactive astrocytes and microglia. Disruption of the CD40R-CD40L signaling also reduces the incidence of Aβ pathology development and the late-stage maturation of β-amyloid plaques.

Tg APP_{sw} mice manifest prominent astrocytosis and microgliosis and develop amyloid deposits comparable to human senile plaques by 16 months of age (Irizarry et al., “APPsw transgenic mice develop age-related A beta deposits and neuropil abnormalities, but no neuronal loss in CA1,” J. Neuropathol. Exp. Neurol. (1997) 56:965-73). To evaluate whether CD40L deficiency might oppose gliosis in Tg APP_{sw} mice, we performed immunohistochemistry for detection of CD11b (a marker of activated microglia) and glial fibrillary acidic protein (GFAP, increased in activated astrocytes). As shown in FIGS. 1a-f, activated microglia appeared to be reduced in Tg APP_{sw}/CD40L def. mice compared to Tg APP_{sw} mice in each of the three brain regions examined (cingulate cortex, hippocampus, and enthorhinal cortex). Quantitative image analysis revealed significant differences for each brain region, showing between 44 and 50% reduction in activated microglia (FIG. 1m). Examination of GFAP-positive astrocytes showed a similar pattern of results, with diminished astrocytic activation ranging from 30 to 46% FIGS. (1g-l, n). Additionally, measurement of brain TNF-α (an activated microglial marker that we have shown is secreted after Aβ and CD40L challenge (Tan et al., “Microglial activation resulting from CD40-CD40L interaction after beta-amyloid stimulation,” Science (1999) 286:2352-55) protein levels by Western immunoblot revealed a statistically significant (p<0.001) 64% reduction in Tg APP_{sw}/CD40L def. mice compared to Tg APP_{sw} mice (mean TNF-α to actin ratio±1 SEM: Tg APP_{sw} mice, 0.247±0.02; control littermates, 0.13±0.01; Tg APP_{sw}/CD40L def. mice, 0.09±0.01; CD40L def. mice, 0.09±0.02), providing further evidence of reduced inflammation in TgAPP_{sw}/CD40L def. mouse brains.

In order to determine if the observed reduction in brain inflammation was associated with diminished Aβ pathology in Tg APPₛw/CD40L def. mice, we evaluated the latter by four strategies: anti-Aβ antibody immunoreactivity (conventional “Aβ burden” analysis), Aβ sandwich enzyme-linked immunoabsorbance assay (ELISA), congo red staining, and Aβ plaque morphometric analysis. While 12-month old Tg APP_{sw} mice had minimal Aβ plaque loads (≦2 plaques per section examined), Aβ plaques were not detectable in age-matched Tg APP_{sw}/CD40L def. mice (data not shown). In 16-month-old mice, up to 51% diminution of Aβ burden was evident in Tg APP_{sw}/CD40L def. mice for the brain regions examined, differences that were statistically significant (mean % ±1 SEM; 41% reduction in cingulate cortex: Tg APP_{sw}, 1.74±0.22; Tg APP_{sw}/CD40L def, 1.02±0.10, p<0.05; 46% reduction in entorhinal cortex: Tg APP_{sw}, 1.12±0.16; Tg APP_{sw}/CD40L def, 0.60±0.06, p<0.001; 51% reduction in hippocampus: Tg APP_{sw}, 0.79±0.08; Tg APP_{sw}/CD40L def., 0.39±0.08, p<0.001). Total Aβ ELISA analysis of these animals produced consistent results [mean Aβ (ng/wet g of brain) ±1 SEM of Tg APP_{sw} mice vs. Tg APP_{sw}/CD40L def. mice; 45% reduction in Aβ₁₋₄₀: 569.01±15.80 vs. 315.04±62.29; 24% reduction in Aβ₁₋₄₂: 469.64±35.20 vs. 355.71±18.85; 35% reduction in total Aβ: 1038.66±21.83 vs. 670.75±81.14]. Analysis of total APP by Western immunoblot did not reveal a significant difference between these mice (mean APP to actin ratio ±1 SEM; Tg APP_{sw} mice, 1.16±0.06; Tg APP_{sw}/CD40L def. mice, 1.15±0.04), suggesting that the observed differences on reduction of Aβ in Tg APP_{sw} mice deficient for CD40L are not due to down-regulation of APP production.

When taken together, our data indicate that blockade of the Aβ-mediated brain inflammatory response by opposing CD40 signaling provides a novel therapeutic target in AD. Additionally, these data support the hypothesis that CD40-mediated brain inflammation is detrimental by promoting Aβ pathology, most likely by affecting microglial activation. The effects reported here on CD40-mediated microgliosis, astrocytosis, and Aβ deposition could also be interpreted within the framework of the CD40-CD40L interaction as a key regulator of the peripheral immune response. As reduction in Aβ load in Tg APP_{sw}/CD40L def. mice was not complete, we hypothesized that interrupting CD40R-CD40L signaling might specifically mitigate formation of the mature, congophillic subset of Aβ plaques. Strikingly, data show between 78 and 86% reduction in congophilic plaques in Tg APP_{sw}/CD40L def. mice (FIG. 2). Morphometric analysis of anti-Aβ antibody immunoreactive Aβ plaques at this age corroborates these data, showing a similar magnitude of reduction in large (>50 μm) and medium-sized (between 25 and 50 μm) Aβ plaque subsets in the neocortices and hippocampi of Tg APP_{sw}/CD40L def. mice (FIG. 3). Similar to a previous finding implicating CD40L as required for the progression of atherosclerotic plaques (Lutgens et al., “Requirement for CD154 in the progression of atherosclerosis,” Nat. Med. (1999) 5:1313-16), the data presented here particularly support a role of the CD40R-CD40L interaction in the late stage maturation of Aβ plaques.

Immunohistochemistry. Standard methods known in the art and not specifically described are generally followed as in Stites et al. (eds), Basic and Clinical Immunology (8th Edition), APPleton & Lange, Norwalk, Conn. (1994) and Johnstone & Thorpe, Immunochemistry in Practice, Blackwell Scientific Publications, Oxford, 1982. General methods in molecular biology: Standard molecular biology techniques known in the art and not specifically described are generally followed as in Sambrook et al., Molecular Cloning: A Laboratory Manual, Cold Springs Harbor Laboratory, New York (1989, 1992), and in Ausubel et al., Current Protocols in Molecular Biology, John Wiley and Sons, Baltimore, Md. (1989).

Mice. CD40L deficient mice are the C57BL/6 background and were constructed as previously described (Xu et al., “Mice deficient for the CD40 ligand,” Immunity (1994) 1:423-31). Tg APP_{sw} mice are the 2576 line crossed with C57B6/SJL as previously described (Hsiao et al., “Age-related CNS disorder and early death in transgenic FVB/N mice overexpressing Alzheimer amyloid precursor proteins,” Neuron (1995) 15:1203-18). We crossed CD40L deficient mice with Tg APP_{sw} transgenic mice and characterized first and second filial offspring by polymerase chain reaction-based genotyping for the mutant APP construct (to examine Tg APP_{sw} status) and neomycin selection vector (to type for CD40L deficiency), followed by Western blot for brain APP and splenic CD40L protein, respectively. The animals that we then studied at 12 and 16 months of age were Tg APP_{sw}/CD40L deficient (Tg APP_{sw}/CD40L def; 12 months: 3 female, 16 months: 3 female/1 male), non-Tg APP_{sw}/CD40L deficient (CD40L def.; 12 months: 3 female, 16 months: 3 female/1 male), Tg APP_{sw}/CD40L wild-type (Tg APP_{sw}; 12 months: 3 female, 16 months: 2 female/1 male), and non-Tg APP_{sw}/CD40L wild-type control littermate mice (Control; 12 months: 3 female, 16 months: 2 female/1 male).

Mice were anesthetized with isofluorane and transcardinally perfused with ice-cold physiological saline containing heparin. Brains were rapidly dissected and quartered using a mouse brain slicer (Muromachi Kikai Co., Tokyo, Japan). The first and second anterior quarters were homogenized for Western blot analyses, and the third and fourth posterior quarters were used for microtome or cryostat sectioning. For microgliosis analysis, brains were quick-frozen at −80° C., and for Aβ immunohistochemistry, congo red staining, and astrocytosis, brains were immersed in 4% paraformaldehyde at 4° C. overnight, and routinely processed in paraffin. Five coronal sections from each brain (5 μm thickness) were cut with a 150 μm interval for these analyses. Immunohistochemical staining was performed in accordance with the manufacturer's instruction using the VECTASTAIN® Elite ABC kit (Vector Laboratories, Burlingame, Calif.), except that, for CD11b staining, a biotinylated secondary mouse IgG absorbed anti-rat antibody was used in place of the biotinylated anti-rabbit antibody that was supplied with the kit. Congo red staining was performed according to standard practice using 10% (w/v) filtered congo red dye cleared with alkaline alcohol, and methyl green was used for counter-staining. The following antibodies were variously employed for immunohistochemical staining: rabbit anti-cow GFAP antibody (1:500; DAKO, Carpinteria, Calif.), rabbit anti-human amyloid-β antibody (1:100; Sigma, Hercules, Mo.) and rat anti-mouse CD11b antibody (1:200; CALTAG LABORATOIRES, Burlingame, Calif.). Images were acquired from an Olympus BX60 microscope with an attached CCD video camera system (Olympus, Tokyo, Japan), and video signal was routed into a Windows 98SE™ PC via an AG5 averaging flame grabber (Scion Corporation, Frederick, Md.) for quantitative analysis using Image-Pro software (Media Cybernetics, MD). Images of five 5 μm sections (150 μm apart) through each anatomic region of interest (hippocampus or cortical areas) were captured and a threshold optical density was obtained that discriminated staining from background. Manual editing of each field was used to eliminate artifacts. For Aβ or congo red burden, astrocytosis and microgliosis analyses, data are reported as the percentage of immunolabeled area captured (positive pixels) divided by the full area captured (total pixels). For Aβ plaque morphometric analysis, diameters of Aβ plaques were calculated via quantitative image analysis and numbers of plaques falling into each diameter category were totaled. Each immunohistochemical analysis was performed by a single examiner (T.M. or T.T.) blinded to sample identities.

Mouse brains (Control, Tg APP_{sw}, CD40L def., and Tg APP_{sw}/CD40L def.) were isolated under sterile conditions on ice and placed in ice-cold lysis buffer (containing 20 mM Tris, pH 7.5, 150 mM NaCl, 1 mM EDTA, 1 mM EGTA, 1% v/v Triton X-100, 2.5 mM sodium pyrophosphate, 1 mM ,β-glycerolphosphate, 1 mM Na₃VO₄, 1 μg/mL leupeptin, and 1 mM PMSF). Brains were then sonicated on ice for approximately 3 min, let stand for 15 min at 4° C., and centrifuged at 15,000 rpm for 15 min. Total Aβ species were detected by acid extraction of brain homogenates in 5 M guanidine buffer (Johnson-Wood et al., “Amyloid precursor protein processing and A beta42 deposition in a transgenic mouse model of Alzheimer disease,” Proc. Natl. Acad. Sci. USA (1997) 94:1550-5), followed by a 1:10 dilution in lysis buffer, and Aβ₁₋₄₀, Aβ₁₋₄₂, and total Aβ (estimated by summing Aβ₁₋₄₀ and Aβ₁₋₄₂ values) were quantified in these samples using the Aβ₁₋₄₀ and AP₁₋₄₂ enzyme-linked immunosorbent assay (ELISA) kits (QCB, Hopkinton, Mass.), in accordance with the manufacturer's instruction, except that standards were diluted such that the final concentration included 0.5 M guanidine buffer. Total protein was quantified in brain homogenates using the Bio-Rad protein assay (Bio-Rad, Hercules, Calif.); thus, ELISA values are reported as ng of Aβ₁₋ₓ/wet g of brain.

All data in this example were found to be normally distributed; therefore, in instances of single mean comparison, Levene's test for equality of variances followed by t-Test for independent samples was used to assess significance. In instances of multiple mean comparisons, analysis of variance (ANOVA) was employed, followed by post-hoc comparison using Bonferroni's method. For all analyses, alpha levels were set at 0.05 and analyses were performed using SPSS for Windows, release 10.0.5.

### EXAMPLE 2

### Exogenous Disruption of CD40L Function

Exogenous disruption of CD40L function was examined for the ability to produce a similar phenotype as genetic ablation in a transgenic mouse model of accelerated cerebral amyloidosis. Animals were treated with anti-CD40L antibody and a comparable reduction of 4G8-positive and thioflavin S-staining β-amyloid plaques were observed. Attenuated Aβ/β-amyloid pathology in both of these scenarios is associated with modulation of APP processing towards the non-amyloidogenic pathway, as the potentially amyloidogenic β-C-terminal fragment (β-CTF) of the amyloid precursor protein (APP) is markedly reduced relative to the α-C-terminal fragment (α-CTF).

We sought to determine the impact of reducing CD40L availability on Aβ/β-amyloid pathology in a mouse model of AD that overproduces Aβ₁₋₄₀ and Aβ₁₋₄₂ and develops significant amyloid deposits by 16 months of age (Tg APP_{sw}, line 2576) (Hsiao et al., “Correlative memory deficits, Abeta elevation, and amyloid plaques in transgenic mice,” Science (1996) 274:99-102). Thus, we crossed Tg APP_{sw} mice with animals deficient in CD40L (TgAPP_{sw}/CD40L def.) (Tan et al., “Microglial activation resulting from CD40-CD40L interaction after beta-amyloid stimulation,” Science (1999) 286:2352-55).

In order to determine if genetic disruption of CD40L could produce diminished Aβ/β-amyloid pathology in Tg APP_{sw}/CD40L def. mice, we evaluated this pathology by four strategies: anti-Aβ antibody immunoreactivity (conventional “β-amyloid burden” analysis), Aβ sandwich enzyme-linked immunoabsorbance assay (ELISA), congo red staining, and β-amyloid plaque morphometric analysis. While 12-month old Tg APP_{sw} mice had minimal β-amyloid plaque loads (≦2 plaques per section examined), β-amyloid plaques were not detectable in age-matched Tg APP_{sw}/CD40L def. mice. Sixteen (16)-month-old TgAPP_{sw} mice had typical β-amyloid load (Irizarry et al., “APPsw transgenic mice develop age-related A beta deposits and neuropil abnormalities, but no neuronal loss in CA1,” Neuropathol. Exp. Neurol. (1997) 56:965-73), up to 51% diminution of β-amyloid burden was evident in Tg APP_{sw}/CD40L def. compared to Tg APP_{sw} mice for the brain regions examined, differences that were statistically significant (mean % ±1 SEM; 41% reduction in cingulate cortex: Tg APP_{sw}, 1.74±0.22; Tg APP_{sw}/CD40L def., 1.02±0.10, p<0.05; 46% reduction in entorhinal cortex: Tg APP_{sw}, 1.12±0.16; Tg APP_{sw}/CD40L def., 0.60±0.06, p<0.001; 51% reduction in the hippocampus: Tg APP_{sw}, 0.79±0.08; Tg APP_{sw}/CD40L def., 0.39±0.08, p<0.001). Aβ ELISA analysis of these animals produced results consistent with the above findings [mean Aβ (ng/wet g of brain) ±1 SEM of Tg APP_{sw} mice vs. Tg APP_{sw}/CD40L def. mice; 45% reduction in Aβ₁₋₄₀: 569.0±15.8 vs. 315.0±62.3; 24% reduction in Aβ₁₋₄₂: 469.6±35.2 vs. 355.7±18.9; 35% reduction in total Aβ: 1038.7±21.8 vs. 670.8±81.1, p<0.001 for each comparison]. Most notably, congophilic β-amyloid deposits were markedly reduced in Tg APP_{sw}/CD40L def. mice, as our data show a 78% (H) to 86% (CC) reduction compared to Tg APP_{sw} mice. In addition, morphometric analysis of anti-Aβ antibody immunoreactive β-amyloid plaques at this age showed a reduction in large (>50 μm) and medium-sized (between 25 and 50 μm) β-amyloid plaque subsets in their neocortices and hippocampi. Analysis of total APP by Western immunoblot did not reveal a significant difference between these mice (mean APP to actin ratio ±1 SEM; Tg APP_{sw} mice, 1.16±0.06; Tg APP_{sw}/CD40L def. mice, 1.15±0.04), suggesting that the observed reduction reduction of Aβ/β-amyloid in Tg APP_{sw}/CD40L def. mice was not due to reduced APP production.

To evaluate whether CD40L deficiency might oppose gliosis in Tg APP_{sw} mice, we performed immunohistochemistry for detection of CD11b (a marker of activated microglia) and glial fibrillary acidic protein (GFAβ, increased in activated astrocytes). Microglial activation was reduced in Tg APP_{sw}/CD40L def. mice compared to Tg APP_{sw} mice in each of the three brain regions examined [cingulate cortex (CC), hippocampus (H), and entorhinal cortex (EC)] by 16 months of age. Quantitative image analysis revealed significant differences for each brain region, showing between 44% (CC) and 50% (EC) reduction in activated microglia. Examination of GFAP-positive astrocytes showed a similar pattern of results, with diminished astrocytic activation ranging from 30% (EC) to 46% (H). Additionally, measurement of brain TNF-α protein [secreted by activated microglia and astrocytes] levels by Western immunoblot revealed a statistically significant (p<0.001) 64% reduction in Tg APP_{sw}/CD40L def. mice compared to Tg APP_{sw} mice (mean TNF-α to actin ratio±1 SEM: Tg APP_{sw} mice, 0.25±0.02; control littermates, 0.13±0.01; Tg APP_{sw}/CD40L def. mice, 0.09±0.01; CD40L def mice, 0.09±0.02), providing further evidence of reduced gliosis in TgAPP_{sw}/CD40L def. mouse brains.

Anti-CD40L antibody was administered to a transgenic mouse model of AD. To expedite evaluation in these experiments, we administered anti-CD40L antibody to mice doubly transgenic for the “Swedish” APP and M146L PS1 mutations (PSAPP). These mice have previously been shown to produce copious β-amyloid deposits by 8 months of age (Holcomb et al., “Accelerated Alzheimer-type phenotype in transgenic mice carrying both mutant amyloid precursor protein and presenilin I transgenes,” Nat. Med. (1998) 4:97-100). Anti-CD40L antibody was administered based on a treatment schedule previously described, which depletes CD40L in mice (Schonbeck et al., “Inhibition of CD40 signaling limits evolution of established atherosclerosis in mice,” Proc. Natl. Acad. Sci. USA (2000) 97:7458-63). At 8 months of age β-amyloid plaques appeared more diffuse in PSAPP mice that received anti-CD40L antibody treatment. Results revealed between 61% (H) and 74% (EC) reduction in β-amyloid plaques in PSAPP mice treated with anti-CD40L antibody versus isotype-matched control antibody. The largest reductions were observed in the hippocampus and entorhinal cortex, regions classically regarded to be most sensitive to AD pathology in humans (Schmidt et al., “Relative abundance of tau and neurofilament epitopes in hippocampal neurofibrillary tangles,” Am. J. Pathol. (1990) 136:1069-75; Ball et al., “A new definition of Alzheimer's disease: a hippocampal dementia,” Lancet (1985) 1:14-16). Consistently, thioflavin S staining for β-amyloid revealed reductions of similar magnitude in these same regions. Thus, either genetic disruption of CD40L from conception or depletion of CD40L in adult transgenic mice results in mitigation of cerebral amyloidosis.

We examined the ratio of β-C-terminal fragment (β-CTF) to at α-C-terminal fragment (α-CTF) of APP in Tg APP_{sw} mice, Tg APP_{sw}/CD40L def. mice, PSAPP animals treated with anti-CD40L antibody, and PSAPP mice treated with non-specific, isotype-matched control antibody. As previously reported, α-CTF and β-CTF were represented at similar levels in Tg APP_{sw} mice in contrast to the largely α-CTF processing of normal APP in murine cells (Luo et al., “Mice deficient in BACE1, the Alzheimer's beta-secretase, have normal phenotype and abolished beta-amyloid generation,” Nat. Neurosci. (2001) 4:231-2). Strikingly, Tg APP_{sw}/CD40L def. animals had a marked decrease of β-CTF relative to α-CTF. In contrast to Tg APP_{sw} mice, in PSAPP animals, α-CTF was under-represented relative to β-CTF in animals that received non-relevant control IgG antibody (IgG-treated PSAPP mice did not differ from non-treated PSAPP animals, data not shown). This is consistent with the generation of excess Aβ/β-amyloid in these animals. By contrast, PSAPP mice that received anti-CD40L antibody manifested a shift in APP CTFs such that the ratio of β-CTF to α-CTF was markedly decreased compared to controls. To establish whether anti-CD40L antibody could penetrate the blood brain barrier and could potentially directly effect changes in CNS APP processing (as opposed to the generation of a peripheral signal or some other mechanism) we probed brain homogenates for hamster IgG antibody and found it to be present at 0.245% of circulating levels after 24 hours (no significant difference was found between anti-CD40L and control antibody, data not shown).

We have recently identified CD40 on neurons and neuron-like cells (including the N2a neuroblastoma cell line), and have shown that neuronal CD40 is functional, being intimately involved in neuronal development, survival, and maturation (Tan et al., “CD40 is expressed and functional on neuronal cells,” EMBO J. (2002) 21:643-52). Given our in vivo findings, we wished to determine whether CD40L could directly act on neurons to modulate APP processing. An N2a cell line was established that stably overexpresses (by˜3-fold) the human wild-type APP-751 transgene (Xia et al., “Enhanced production and oligomerization of the 42-residue amyloid beta-protein by Chinese hamster ovary cells stably expressing mutant presenilins,” J. Biol. Chem. (1997) 272:7977-82). CD40L treatment of these cells results in a time-dependent decrease in α-CTF by Western blot. To confirm whether this reduction in α-CTF might be associated with amyloidogenic processing of APP, we measured secreted Aβ in conditioned media. Results show a time-dependent increase in both Aβ₁₋₄₀ and Aβ₁₋₄₂ levels, which is inversely related to α-CTF levels. Thus, CD40L is able to directly promote amyloidogenic APP processing in neurons or neuron-like cells. Reducing the availability of CD40L in vivo has the opposite effect of adding CD40L in vitro on APP processing, both suggesting that CD40L regulates secretase cleavage of APP. As the vast majority of cases of AD are associated with accumulation of Aβ from a normal APP sequence, the observation that the processing of normal APP can be pushed towards amyloidogenicity by CD40L is of interest. In AD, it has been observed that an excess of CD40L-bearing astrocytes occurs (Calingasan et al., “Identification of CD40 ligand in Alzheimer's disease and in animal models of Alzheimer's disease and brain injury,” Neurobiol. Aging (2002) 23:31-9), and either membrane-bound or secreted forms of CD40L (Schonbeck et al., “The CD40/CD154 receptor/ligand dyad,” Cell Mol. Life Sci. (2001) 58:4-43) could influence cerebral APP processing towards Aβ formation.

Mice. CD40L deficient mice are the C57BL/6 background constructed as previously described (Xu et al., “Mice deficient for the CD40 ligand,” Immunity (1994) 1:423-31). Tg APP_{sw} mice are the 2576 line crossed with C57B6/SJL as previously described (Hsiao et al., “Correlative memory deficits, Abeta elevation, and amyloid plaques in transgenic mice,” Science (1996) 274:99-102). Also, CD40L deficient mice were crossed with Tg APP_{sw} transgenic mice and characterized offspring by polymerase chain reaction-based genotyping for the mutant APP construct (to examine Tg APP_{sw} status) and neomycin selection vector (to type for CD40L deficiency), followed by Western blot for brain APP and splenic CD40L protein, respectively. The animals that we studied at 12 and 16 months of age were Tg APP_{sw}/CD40L deficient (Tg APP_{sw}/CD40L def.; 12 months: 3 female, 16 months: 3 female/1 male), non-Tg APP_{sw}/CD40L deficient (CD40L def.; 12 months: 3 female, 16 months: 3 female/1 male), Tg APP_{sw}/CD40L wild-type (Tg APP_{sw}; 12 months: 3 female, 16 months: 2 female/1 male), and non-Tg APP_{sw}/CD40L wild-type control littermate mice (Control; 12 months: 3 female, 16 months: 2 female/1 male).

PSAPP were bred by crossing Tg APP_{sw} with PS1 M1467 mice as previously described (Holcomb et al., “Accelerated Alzheimer-type phenotype in transgenic mice carrying both mutant amyloid precursor protein and presenilin 1 transgenes,” Nat. Med. (1998) 4:97-100). A total of 10 PSAPP mice were used in this study, and 5 mice (3 female/2 male) received anti-CD40L IgG antibody (MR1), while the remaining 5 (2 female/3 male) received isotype-matched control IgG antibody. Beginning at 8 weeks of age, PSAPP mice were i.p. injected with 200 μg of the appropriate antibody once every ten days, based on previously described methods (Schonbeck et al., “Inhibition of CD40 signaling limits evolution of established atherosclerosis in mice,” Proc. Natl. Acad. Sci. USA (2000) 97:7458-63). These mice were then sacrificed at 8 months of age for analysis of Aβ deposits.

Mice were anesthetized with isofluorane and transcardinally perfused with ice-cold physiological saline containing heparin. Brains were rapidly dissected and quartered using a mouse brain slicer (Muromachi Kikai Co., Tokyo). The first and second anterior quarters were homogenized for Western blot analyses, and the third and fourth posterior quarters were used for microtome or cryostat sectioning. For microgliosis analysis, brains were quick-frozen at −80° C., and for β-amyloid immunohistochemistry, congo red staining, and astrocytosis, brains were immersed in 4% paraformaldehyde at 4° C. overnight, and routinely processed in paraffin. Five coronal sections from each brain (5 μm thickness) were cut with a 150 μm interval for these analyses. Immunohistochemical staining was performed in accordance with the manufacturer's instruction using the VECTASTAIN® Elite ABC kit (Vector Laboratories), except that, for CD11b staining, a biotinylated secondary mouse IgG absorbed anti-rat antibody was used in place of the biotinylated anti-rabbit antibody that was supplied with the kit. Congo red staining was performed according to standard practice using 10% (w/v) filtered congo red dye cleared with alkaline alcohol. The following antibodies were variously employed for immunohistochemical staining: rabbit anti-cow GFAP antibody (1:500; DAKO), mouse anti-human amyloid-β antibody (4G8; 1:100; Signet), rabbit anti-human amyloid-β antibody (1:100; Sigma), and rat anti-mouse CD11b antibody (1:200; Caltag Laboratories).

Image analysis. Images were acquired from an Olympus BX60 microscope with an attached CCD video camera system (Olympus), and video signal was routed into a Windows 98SE™ PC via an AG5 averaging frame grabber (Scion Corporation) for quantitative analysis using Image-Pro software (Media Cybernetics). Images of five 5 μm sections (150 μm apart) through each anatomic region of interest (hippocampus or cortical areas) were captured and a threshold optical density was obtained that discriminated staining from background. Manual editing of each field was used to eliminate artifacts. For β-amyloid, congo red, and thioflavin S burden, and astrocytosis and microgliosis analyses, data are reported as the percentage of immunolabeled area captured (positive pixels) divided by the full area captured (total pixels). For β-amyloid plaque morphometric analysis, diameters of β-amyloid plaques were calculated via quantitative image analysis and numbers of plaques falling into each diameter category were totaled. Each immunohistochemical analysis was performed by a single examiner (T.M. or T.T.). Image analysis was performed prior to the revelation of sample identities.

ELISA analysis. Mouse brains (Control, Tg APP_{sw}, CD40L def., and Tg APP_{sw}/CD40L def.) were isolated under sterile conditions on ice and placed in ice-cold lysis buffer (containing 20 mM Tris, pH 7.5, 150 mM NaCl, 1 mM EDTA, 1 mM EGTA, 1% v/v Triton X-100, 2.5 mM sodium pyrophosphate, 1 mM β-glycerolphosphate, 1 mM Na₃VO₄, 1 μg/mL leupeptin, and 1 mM PMSF). Brains were then sonicated on ice for approximately 3 min, let stand for 15 min at 4° C., and centrifuged at 15,000 rpm for 15 min. Total Aβ species were detected by acid extraction of brain homogenates in 5 M guanidine buffer (Johnson-Wood et al., “Amyloid precursor protein processing and A beta42 deposition in a transgenic mouse model of Alzheimer disease,” Proc. Natl. Acad. Sci. USA (1997) 94:1550-55), followed by a 1:10 dilution in lysis buffer. Aβ₁₋₄₀, Aβ₁₋₄₂, and total Aβ (estimated by summing Aβ₁₋₄₀ and Aβ₁₋₄₂ values) were quantified in these samples using the Aβ₁₋₄₀ and Aβ₁₋₄₂ enzyme-linked immunosorbent assay (ELISA) kits (QCB) in accordance with the manufacturer's instruction, except that standards were diluted such that the final concentration included 0.5 M guanidine buffer. Total protein was quantified in brain homogenates using the Bio-Rad protein assay (Bio-Rad); thus, ELISA values are reported as ng of Aβ₁₋ₓ/wet g of brain. For in vitro analysis of Aβ levels, conditioned media from human APP-overexpressing N2a cells was collected and analyzed at a 1:1 dilution using the method described above, and values were reported as percentage of Aβ₁₋ₓ secreted relative to control.

Western blot. Mouse brains or cells were lysed in ice-cold lysis buffer as described above, and an aliquot corresponding to 50 μg of total protein was electrophoretically separated using 16.5% Tris-tricine gels (Bio-Rad, Hercules, Calif.). Electrophoresed proteins were then transferred to PVDF membranes (Bio-Rad), washed in dH₂0, and blocked for 1 h at ambient temperature in Tris-buffered saline (TBS) containing 5% (w/v) of non-fat dry milk. After blocking, membranes were hybridized for 1 h at ambient temperature with various antibodies against the C-terminus of APP or the N-terminus of Aβ. Membranes were then washed 3× for 5 min each in dH₂0 and incubated for 1 h at ambient temperature with the appropriate HRP-conjugated secondary antibody (1:1000, Santa Cruz Biotechnology, Santa Cruz, Calif.). All antibodies were diluted in TBS containing 5% (w/v) of non-fat dry milk. Blots were developed using the luminol reagent (Santa Cruz). Densitometric analysis was performed using the Fluor-S MultiImager™ with Quantity One™ software (Bio-Rad). Antibodies used for Western blot included antibody 369 (1:500, kindly provided by Dr. Sam Gandy), 6687 (1:1,000, kindly provided by Dr. Harald Steiner), Chemicon anti-C-terminal APP antibody (1:500), BAM-10 (1:1000, Sigma), or actin (as an internal reference control, 1:1000, Roche, Germany).

Statistical analyses. All data for this example were found to be normally distributed; therefore, in instances of single mean comparison, Levene's test for equality of variances followed by t-Test for independent samples was used to assess significance. In instances of multiple mean comparisons, analysis of variance (ANOVA) was employed, followed by post-hoc comparison using Bonferroni's method. For all analyses, alpha levels were set at 0.05 and were performed using SPSS for Windows, release 10.0.5.

### EXAMPLE 3

### Detection of Phospho-tau in Mouse Brain Sections

Immunohistochemistry. Transgenic mice [16 m old, including Tg APP_{sw} mice: n=4, 2 male/2 female, and Tg APP_{sw}/CD40L def. mice: n=5, 3 female, 2 male] were anesthetized with isofluorane and transcardinally perfused with ice-cold physiological saline containing heparin. Brains were rapidly dissected and immersed in 4% paraformaldehyde at 4° C. overnight. Brain tissue was routinely embedded in paraffin and processed according to standard practice. Five coronal sections (5 μm thickness) were cut with a 150 μm interval using a Reichert-Jung 2030 microtome (Leica Co., Nussloch, Germany). Immunohistochemical staining was performed in accordance with the manufacturer's instruction using the VECTASTAIN® Elite avadin biotin complex (ABC) kit (Vector Laboratories, Burlingame, Calif.). The primary antibodies that were employed were anti-phospho-tau S199 (1:50) and anti-phospho-tau S202 (1:200) (both antibodies were obtained from BioSource International, Camarillo, Calif.). Slides were permanently mounted and viewed under bright-field using an Olympus BX-60 microscope.

Image analysis. Bright-field images were acquired from an Olympus BX-60 microscope with an attached MagnaFire™ camera, and video signal was routed into a Windows 98SE™ PC for quantitative analysis using Image-Pro software (Media Cybernetics, Silver Spring, Md.). Images of five 5 μm sections (150 μm apart) through each anatomic region of interest (hippocampus or cortical areas) were captured and a threshold optical density was obtained that discriminated staining from background. Manual editing of each field was used to eliminate artifacts. Positive immunolabeled area was determined by dividing the percentage of immunolabeled area captured (positive pixels) by the full area captured (total pixels). Image analysis was performed in a blind fashion prior to the revelation of sample identities.

Results. Phosphorylation of tau was examined in situ at 16 m of age in these mice using antibodies that recognize epitopes which are phosphorylated in AD brain (Genis et al., 1999). Antibody pS 199 revealed numerous positive neurons, particularly in close vicinity of β-amyloid deposits in the neocortex and hippocampus of Tg APP_{sw} mice. Yet, in similar regions of Tg APP_{sw}/CD40L def. mouse brains, this neuronal signal was either completely absent or markedly reduced. Quantitative image analysis of multiple brain sections revealed an 83% reduction in neocortical pS199 immunostaining, and a 70% reduction in hippocampal pS199 immunoreactivity. The t-Test for independent samples revealed significant differences between Tg APP_{sw} and Tg APP_{sw}/CD40L def. mice for the neocortex (p<0.01) and the hippocampus (p<0.05). Immunostaining was also performed using antibody pS202. The pattern of immunoreactivity for this antibody was quite different from that of pS199, as pS202 revealed a punctate staining pattern within the area delineated by the β-amyloid deposit, while pS202 positive neurons surrounding the β-amyloid deposit were few in number in both the neocortex and the hippocampus of Tg APP_{sw} mice. When comparing Tg APP_{sw} mice to Tg APP_{sw}/CD40L def. animals, pS202 immunoreactivity was markedly reduced in the latter group. Quantitative image analysis of multiple brain sections revealed a 95% reduction in neocortical pS202 immunostaining, and an 86% reduction in hippocampal pS202 immunoreactivity. The t-Test for independent samples revealed significant differences between Tg APP_{sw} and Tg APP_{sw}/CD40L def. mice for the neocortex (p<0.01) and the hippocampus (p<0.05). Phospho-tau as detected by pS199 or pS202 antibody was essentially absent in Tg APP_{sw} control littermates or CD40L def. mice (data not shown).

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method of identifying compounds that modulate the CD40 ligand/CD40 receptor (CD40L/CD40R) signaling pathway comprising contacting a first sample of cells expressing CD40 receptor (CD40R) with CD40 ligand (CD40L) and measuring a marker; contacting a second sample of cells expressing CD40R with a compound and CD40 ligand, and measuring said marker; and comparing said marker of said first sample of cells with said marker of said second sample of cells.

2. The method of claim 1, wherein said cells are central nervous system (CNS) cells, cell lines derived from central nervous system (CNS) cells, peripheral cells, cell lines derived from peripheral cells, transgenic cells, transgenic cells derived from transgenic animals, or human cells or cell lines.

3. A method of identifying compounds that modulate the CD40L/CD40R signaling pathway comprising:
a. contacting CNS cells expressing CD40R with CD40 ligand and a compound and measuring a marker;
b. contacting peripheral cells expressing CD40R with CD40 ligand and said compound and measuring a marker;
c. contacting CNS cells with a stimulator of the CD40L/CD40R signaling pathway and a compound and measuring a marker;
d. contacting peripheral cells with a stimulator of the CD40L/CD40R signaling pathway and said compound and measuring a marker;
e. contacting CNS cells with an inhibitor of the CD40L/CD40R signaling pathway and said compound and measuring a marker;
f. contacting peripheral cells with an inhibitor of the CD40L/CD40R signaling pathway and said compound and a marker; and
g. comparing said markers to identify those compounds that modulate the CD40L/CD40R signaling pathway.

4. The method of claim 1, wherein the marker is the levels or amounts of one or more cytokine.

5. The method of claim 4, wherein said cytokine is selected from the group consisting of tumor necrosis factor, interleukin 1, interleukin 6, interleukin 12, interleukin 18, macrophage inflammatory protein, macrophage chemoattractant protein, granulocyte-macrophage colony stimulating factor, macrophage colony stimulating factor, and combinations thereof.

6. The method of claim 1, wherein the marker is selected from the group consisting of levels, amounts, or activities of glutamate release, nitric oxide production, nitric oxide synthase, superoxide, superoxide dismutase, and combinations thereof.

7. The method of claim 1, wherein the marker is selected from the group consisting of a major histocompatibility complex molecule, CD45, CD11b, F4/80 antigen, integrins, a cell surface molecule, or combinations thereof.

8. The method of claim 1, wherein the marker is the levels or amounts of Aβ, β-amyloid precursor protein, a fragment of a β-amyloid precursor protein, a fragment of Aβ, or combinations thereof.

9. The method of claim 2 in which said stimulator is an agonistic antibody.

10. The method of claim 2 in which said inhibitor is an antagonistic antibody.

11. The method according to claim 1, wherein said compound binds to CD40L or decreases trimerization of CD40R.

12. The method according to claim 1, wherein said compound binds to CD40R or decreases trimerization of CD40R.

13. The method according to claim 1, wherein said compound modulates the CD40L/CD40R signaling pathway upstream or downstream of CD40L/CD40R interaction.

14. The method according to claim 2, wherein said compound binds to CD40L.

15. The method according to claim 2, wherein said compound binds to CD40R.

16. The method according to claim 2, wherein said compound modulates the CD40L/CD40R signaling pathway downstream or upstream of CD40L/CD40R interaction.

17. A method of identifying compounds that reduce, ameliorate, or modulate symptoms associated with neuronal inflammation, brain injury/trauma, tauopathies, or amyloidogenic diseases comprising administering a compound that modulates the CD40L/CD40R signaling pathway to an animal model and measuring or observing the reduction, amelioration, or modulation of said symptoms.

18. The method according to claim 17, wherein said amyloidgenic diseases are selected from the group consisting of scrapie, transmissible spongioform encephalopathies (TSE's), hereditary cerebral hemorrhage with amyloidosis Icelandic-type (HCHWA-I), hereditary cerebral hemorrhage with amyloidosis Dutch-type (HCHWA-D), familial Mediterranean fever, familial amyloid nephropathy with urticaria and deafness (Muckle-Wells syndrome), myeloma or macroglobulinernia-associated idopathy associated with amyloid, familial amyloid polyneuropathy (Portuguese), familial amyloid cardiomyopathy (Danish), systemic senile amyloidosis, familial amyloid polyneuropathy (Iowa), familial amyloidosis (Finnish), Gerstmann-Staussler-Scheinker syndrome, medullary carcinoma of thyroid, isolated atrial amyloid, Islets of Langerhans, diabetes type II, and insulinoma.

19. The method according to claim 17, wherein said symptoms are selected from the group consisting of reductions in the size and/or number of amyloid plaques, reduction in β-amyloid burden, reduction in soluble Aβ levels, reduction in total Aβ levels, reduction of congophilic β-amyloid deposits, reduction of reactive gliosis, microgliosis, astrocytosis and combinations of said symptoms.

20. A method of treating neuronal inflammation, brain injury/trauma, tauopathies, or amyloidogenic diseases comprising the administration, to an individual, of therapeutically effective amounts of a composition comprising a carrier and an agent that interferes with CD40L/CD40R signaling pathway or the phosphorylation of tau protein.

21. The method according to claim 20, wherein said agent is selected from the group consisting of CD40 ligand (CD40L), soluble CD40L, immunogenic CD40L, CD40L variants (CD40LV), antibodies that bind to CD40L and block its interaction with CD40R, antibodies that bind to CD40R and block ligand binding to the receptor, soluble CD40LV that bind to CD40R and fails to activate the receptor, interfering RNA or antisense RNA to CD40R, or CD40L, and combinations of said agents.

22. The method according to claim 20, wherein said amyloidogenic diseases are selected from the group consisting of scrapie, transmissible spongioform encephalopathies (TSE's), hereditary cerebral hemorrhage with amyloidosis Icelandic-type (HCHWA-I), hereditary cerebral hemorrhage with amyloidosis Dutch-type (HCHWA-D), familial Mediterranean fever, familial amyloid nephropathy with urticaria and deafness (Muckle-Wells syndrome), myeloma or macroglobulinernia-associated idopathy associated with amyloid, familial amyloid polyneuropathy (Portuguese), familial amyloid cardiomyopathy (Danish), systemic senile amyloidosis, familial amyloid polyneuropathy (Iowa), familial amyloidosis (Finnish), Gerstmann-Staussler-Scheinker syndrome, medullary carcinoma of thyroid, isolated atrial amyloid, Islets of Langerhans, diabetes type II, and insulinoma.

23. The method according to claim 2, wherein said transgenic animal is a transgenic worm, transgenic fly, or transgenic rodent.

24. The method according to claim 17 wherein said tauopathies are selected from the group consisting of frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, disinhibition-dementia-parkinsonism-amytrophy complex, Pick's disease, or Pick's disease-like dementia.

25. The method according to claim 20 wherein said tauopathies are selected from the group consisting of frontotemporal dementia, frontotemporal dementia with Parkinsonism, frontotemporal lobe dementia, pallidopontonigral degeneration, progressive supranuclear palsy, multiple system tauopathy, multiple system tauopathy with presenile dementia, Wilhelmsen-Lynch disease, disinhibition-dementia-parkinsonism-amytrophy complex, Pick's disease, or Pick's disease-like dementia.
